(19) United States
(12) Patent Application Publication
(10) Pub. No.: US 2024/0193785 A1
(43) Pub. Date: Jun. 13, 2024

(54) MEDICAL IMAGE PROCESSING APPARATUS, HEPATIC SEGMENT DIVISION METHOD, AND PROGRAM

(71) Applicant: FUJIFILM Corporation, Tokyo (JP)

(72) Inventors: Kiyoshi HASEGAWA, Tokyo (JP); Yusuke KAZAMI, Tokyo (JP); Junichi KANEKO, Tokyo (JP); Deepak KESHWANI, Tokyo (JP)

(73) Assignee: FUJIFILM Corporation, Tokyo (JP)

(21) Appl. No.: 18/587,853

(22) Filed: Feb. 26, 2024

Related U.S. Application Data
(63) Continuation of application No. PCT/JP2022/027537, filed on Jul. 13, 2022.

(30) Foreign Application Priority Data
Aug. 31, 2021 (JP) 2021-141653

(51) Int. Cl. G06T 7/11; G06V 10/764; G06V 10/774; G06V 10/776; G06V 10/82; G06V 20/70; G16H 30/40

(52) U.S. Cl. CPC G06T 7/11; G06V 10/764; G06V 10/774; G06V 10/776; G06V 10/82; G06V 20/70; G16H 30/40; G06T 2200/04; G06T 2207/10081; G06T 2207/10088; G06T 2207/20081; G06T 2207/20084; G06T 2207/30056; G06T 2207/30101; G06V 2201/031

(57) ABSTRACT

A medical image processing apparatus employs a trained model generated by performing machine learning using training data that includes first input data including a first image regarding a liver, and portal vein branch labeling data in which a portal vein branch label is attached to a portal vein region in the liver in the first image for each portal vein branch corresponding to a hepatic segment. The medical image processing apparatus uses the trained model to assign the portal vein branch label to each image unit element of a second image region including at least a liver region of the second image included in a second input data which is the same type as the first input data, and divides the liver region included in the second input data into hepatic segments based on the portal vein branch label assigned to each image unit element of the second image region.

### CROSS-REFERENCE TO RELATED APPLICATIONS

The present application is a Continuation of PCT International Application No. PCT/JP2022/027537 filed on Jul. 13, 2022 claiming priority under 35 U.S.C § 119(a) to Japanese Patent Application No. 2021-141653 filed on Aug. 31, 2021. Each of the above applications is hereby expressly incorporated by reference, in its entirety, into the present application.

### BACKGROUND OF THE INVENTION

### 1. Field of the Invention

The present disclosure relates to a medical image processing apparatus, a hepatic segment division method, and a program, and particularly relates to machine learning technology and image processing technology that handle medical images in which a region including a liver is imaged.

### 2. Description of the Related Art

The liver is divided into eight segments, S1 to S8, using the branched portal vein as an index. That is, S1 is the caudate lobe, S2 is the lateral posterior segment of the left lobe (dorsolateral segment), S3 is the lateral anterior segment of the left lobe (ventrolateral segment), S4 is the medial segment of the left lobe (quadrate lobe), S5 is the anteroinferior segment of the right lobe, S6 is the posteroinferior segment of the right lobe, S7 is the posterosuperior segment of the right lobe, and S8 is the anterosuperior segment of the right lobe.

It is medically important to divide the liver into anatomical segments, and division into segments from S1 to S8 is required in various situations. For example, in an interpretation report, it is necessary to appropriately specify a hepatic segment on a medical image in a case of reporting a segment where an abnormal tumor is present.

JP2003-033349A and R Beichel et al., “Liver segment approximation in CT data for surgical resection planning”, Medical Imaging 2004: Image Processing. Edited by Fitzpatrick, J. Michael; Sonka, Milan, 2004, Proceedings of the SPIE, Volume 5370, pp. 1435-1446 disclose a method of extracting blood vessels in the liver region, specifying which blood vessel's dominant region a region other than the blood vessels within the liver region (liver parenchyma or the like) belongs to, using a Voronoi diagram, and thereby specifying the dominant region of each blood vessel as the hepatic segment.

In addition, WO2020/203552A discloses a convolutional neural network (CNN) that uses deep learning to perform a class classification task of vascular branches in the liver.

### SUMMARY OF THE INVENTION

The segments from S1 to S8 in the liver do not have clear physical or anatomical boundary surfaces, and there are large individual differences in the positions at which the hepatic segments are divided depending on the person making decision. Therefore, a method for automatically and uniquely dividing the hepatic segments from medical images is desired.

In the related art, the method of dividing the liver region into the segments from S1 to S8 is basically based on each labeled portal vein branch (partial portal vein) within the liver. Specifically, a portal vein region is extracted from the medical image in which a region including the liver is imaged, and the portal vein branches are labeled. Thereafter, Voronoi division is performed based on the distance from the labeled portal vein branch, and the dominant region is set based on the obtained results.

The portal vein is classified and labeled into portal vein branches from S1 to S8, corresponding to the hepatic segments from S1 to S8. For example, the dominant region of the S1 portal vein branch is the S1 hepatic segment, and there may be a one-to-one correspondence between labels of the portal vein branches and labels of the hepatic segments. In the case of Voronoi division, the hepatic segment to which each voxel belongs is decided on the basis of the criterion of which portal vein branch label region each voxel in a three-dimensional image is closest to. The portal vein branch label is a label for classifying (dividing) a predetermined image region into eight regions in association with the eight portal vein branches S1 to S8. Here, the portal vein branch label classifies the portal vein region as a predetermined image region into eight portal vein branch regions, and the hepatic segment to which each voxel belongs is decided on the basis of the distance to the eight portal vein branch regions.

However, the boundary surface of each of the hepatic segments from S1 to S8 is not simple. Therefore, it is difficult for a doctor who is a user to uniquely set a valid boundary surface of the hepatic segment. In addition, it is difficult to automate complicated processing performed by doctors as it is.

In addition, some images captured by the modality have a low density value of the voxel in the portal vein region, or a portal vein region is not properly imaged in the image. In particular, there are images in which the terminal portion of the portal vein is not shown. As a result, the method using the Voronoi division based on the specified portal vein region may not be able to accurately divide the hepatic segments. That is, in the method using the Voronoi division, the accuracy of the division of the hepatic segments changes depending on how blood vessels (portal veins) are shown in the image (refer to FIGS. 12 and 13).

For such a problem, a method is considered which uses machine learning to generate a learning model that performs a division task of the hepatic segments. That is, as training data, a large number of data sets of input images and data with the ground truth label for each of the hepatic segments S1 to S8 attached to the input images are prepared, and these data sets are used to perform supervised learning. Thus, a trained model that outputs division results of the hepatic segments is generated.

However, in the above method, it is necessary for the doctor to perform a labeling work of assigning a ground truth label for each hepatic segment to the input image. Preparing the ground truth label for each of hepatic segments S1 to S8 for a large number of images places an enormous burden on the doctor. Furthermore, in a case of labeling the hepatic segments, there are individual differences among doctors, and it is difficult to prepare uniform ground truth data (teaching data). In order to achieve a desired task using machine learning, a method is required which reduces a work load of doctors and the like in a case of generating training data to relatively easily prepare a large amount of training data with high quality.

The present disclosure has been made in view of such circumstances, and an object thereof is to provide a medical image processing apparatus, a hepatic segment division method, and a program which can accurately perform segment division of the liver from medical images.

A medical image processing apparatus according to an aspect of the present disclosure comprising: a processor; and a storage device that stores a program to be executed by the processor, in which the program includes a trained model generated by performing machine learning using training data that includes first input data including a first image regarding a liver, and portal vein branch labeling data in which a portal vein branch label is attached to a portal vein region in the liver in the first image for each portal vein branch corresponding to a hepatic segment, the trained model is a model obtained by updating parameters of a learning model trained to output a labeling result of the portal vein branch label for each image unit element of a first image region of the first image by accepting an input of the first input data, and the processor executes a command of the program to accept second input data which is a same type of input data as the first input data and includes a second image regarding the liver, assign the portal vein branch label to each image unit element of a second image region of the second image using the trained model, and divide a liver region included in the second input data into a plurality of the hepatic segments on the basis of the portal vein branch label assigned to each image unit element of the second image region.

According to the present aspect, it is possible to accurately perform the division of the hepatic segments regardless of how blood vessels are shown in the processing target image. In addition, the portal vein branch labeling data used in the learning for generating the trained model of the present aspect can be generated relatively easily without placing an excessive work load on the doctor. The image unit element in the three-dimensional image may be understood as a voxel, and the image unit element in the two-dimensional image may be understood as a pixel.

In the medical image processing apparatus according to another aspect of the present disclosure, the first input data may include at least one of a computed tomography (CT) image in which a region including the liver is imaged or a portal vein mask image in which a portal vein region is specified, and the first image may be the CT image or the portal vein mask image.

In the medical image processing apparatus according to another aspect of the present disclosure, the first input data may include the CT image and the portal vein mask image.

In the medical image processing apparatus according to another aspect of the present disclosure, the first input data may further include at least one of a liver mask image in which a liver region is specified, a vein mask image in which a vein region is specified, or an inferior vena cava mask image in which an inferior vena cava region is specified.

In the medical image processing apparatus according to another aspect of the present disclosure, the first input data may include the portal vein mask image, the liver mask image, and the vein mask image.

In the medical image processing apparatus according to another aspect of the present disclosure, the first image region may be an entire region of the first image, and the second image region may be an entire region of the second image.

In the medical image processing apparatus according to another aspect of the present disclosure, the portal vein branch label may be a label for classifying the portal vein branch into eight classes corresponding to eight types of the hepatic segments from S1 to S8.

In the medical image processing apparatus according to another aspect of the present disclosure, the trained model may be configured using a convolutional neural network.

In the medical image processing apparatus according to another aspect of the present disclosure, processing of the machine learning for generating the trained model may include calculating a loss only for a portal vein region in which the portal vein branch label is attached, in the portal vein branch labeling data corresponding to the first input data, for a score map indicating a probability of the portal vein branch label output from the learning model, and updating the parameters of the learning model on the basis of the calculated loss.

In the medical image processing apparatus according to another aspect of the present disclosure, each of the first image and the second image may be a three-dimensional image.

In the medical image processing apparatus according to another aspect of the present disclosure, the processor may perform labeling of a hepatic segment label indicating the hepatic segment on the basis of the portal vein branch label assigned to each image unit element of the second image region.

In the medical image processing apparatus according to another aspect of the present disclosure, the second input data may include a CT image in which a region including the liver is imaged, and the processor may extract a liver region from the CT image included in the second input data, and invalidate label information labeled for a region other than the extracted liver region, in the second image region.

Invalidating the label information includes, for example, concepts such as deleting the label information, or ignoring the label information.

In the medical image processing apparatus according to another aspect of the present disclosure, the processor may generate a hepatic segment division image in which a region is divided into the hepatic segments, by converting the portal vein branch label assigned to each image unit element of the second image region into the hepatic segment label.

A hepatic segment division method according to another aspect of the present disclosure is a hepatic segment division method of allowing a computer to divide a liver region in an image into hepatic segments, and comprising: generating a learning model generated by performing machine learning using training data that includes first input data including a first image regarding a liver, and portal vein branch labeling data in which a portal vein branch label is attached to a portal vein region in the liver in the first image for each portal vein branch corresponding to the hepatic segment; generating a trained model by updating parameters of the learning model on the basis of a labeling result of the portal vein branch label that is output by the learning model for each image unit element of a first image region of the first image; accepting second input data which is a same type of input data as the first input data and includes a second image regarding the liver; assigning the portal vein branch label to each image unit element of a second image region of the second image using the trained model; and dividing a liver region included in the second input data into a plurality of the hepatic segments on the basis of the portal vein branch label assigned to each image unit element of the second image region.

A program according to another aspect of the present disclosure is a program that causes a computer to operate as a medical image processing apparatus, and comprising: a trained model generated by performing machine learning using training data that includes first input data including a first image regarding a liver, and portal vein branch labeling data in which a portal vein branch label is attached to a portal vein region in the liver in the first image for each portal vein branch corresponding to a hepatic segment, in which the trained model is a learning model trained to output a labeling result of the portal vein branch label for each image unit element of a first image region including at least a liver region, of the first image by accepting an input of the first input data. The program causes the computer to accept second input data which is a same type of input data as the first input data and includes a second image regarding the liver, assign the portal vein branch label to each image unit element of a second image region of the second image included in the second input data using the trained model, and divide a liver region included in the second input data into a plurality of the hepatic segments on the basis of the portal vein branch label assigned to each image unit element of the second image region.

According to the present disclosure, it is possible to accurately perform the segment division of the liver from a medical image.

### BRIEF DESCRIPTION OF THE DRAWINGS

FIG. 1 is a block diagram illustrating an example of an image processing apparatus that performs processing of generating training data.

FIG. 2 is a block diagram illustrating an example of an information processing apparatus that performs labeling of portal vein branches in a portal vein region.

FIG. 3 is a conceptual diagram illustrating an example of a training data set stored in a training data storage unit.

FIG. 4 is a conceptual diagram illustrating an outline of a learning phase in a case of generating a trained model to be applied to a medical image processing apparatus according to a first embodiment.

FIG. 5 is a block diagram illustrating a configuration example of a learning device.

FIG. 6 is a flowchart illustrating a flow of learning processing of a learning device.

FIG. 7 is a conceptual diagram illustrating an outline of processing in an inference phase using a trained model of the first embodiment.

FIG. 8 is a block diagram illustrating a configuration of the medical image processing apparatus according to the first embodiment.

FIG. 9 is a flowchart illustrating an example of a hepatic segment division method using the medical image processing apparatus according to the first embodiment.

FIG. 10 is a conceptual diagram illustrating an outline of a learning phase in a second embodiment.

FIG. 11 is a block diagram illustrating an outline of an inference phase using a trained model generated by a learning method of the second embodiment.

FIG. 12 is an image example illustrating an example of a hepatic segment division method using Voronoi division according to a comparative example.

FIG. 13 is a diagram illustrating a comparison between a processing result of hepatic segment division based on the Voronoi division according to the comparative example and a result of proper hepatic segment division.

### DESCRIPTION OF THE PREFERRED EMBODIMENTS

Hereinafter, preferred embodiments of the present invention will be described in detail with reference to the accompanying drawings.

### Outline of First Embodiment

Here, a CT image obtained by imaging a region including a liver of a patient using a CT device will be described as an example. There is a problem in that it is a heavy burden for the doctor to create the label for each segment of the liver (ground truth label for the hepatic segment) on the CT image in which a region including the liver is imaged, and there are large individual differences in labeling.

On the other hand, labeling the portal vein branch regions by type in the portal vein region within the liver does not place much of a burden on the doctor compared to the work for labeling the hepatic segment, and regarding the result of the labeling, there is little variation in the decision among doctors.

From this background, in the first embodiment of the present disclosure, machine learning is performed using image data in which the portal vein region is labeled according to the type of portal vein branch, as one of the training data. In addition, the trained model obtained as a result of machine learning is used to realize the segment division of the liver (segmentation of hepatic segment). That is, the training data is data used for a learning model 50, which will be described later, to perform machine learning. In addition, a trained model 650 is generated by subjecting the learning model 50 to machine learning using the training data. That is, the trained model 650 is a model in which parameters of the learning model 50 are optimized. The trained model 650 is applied to a medical image processing apparatus 70 according to the first embodiment.

### Preparation of Training Data

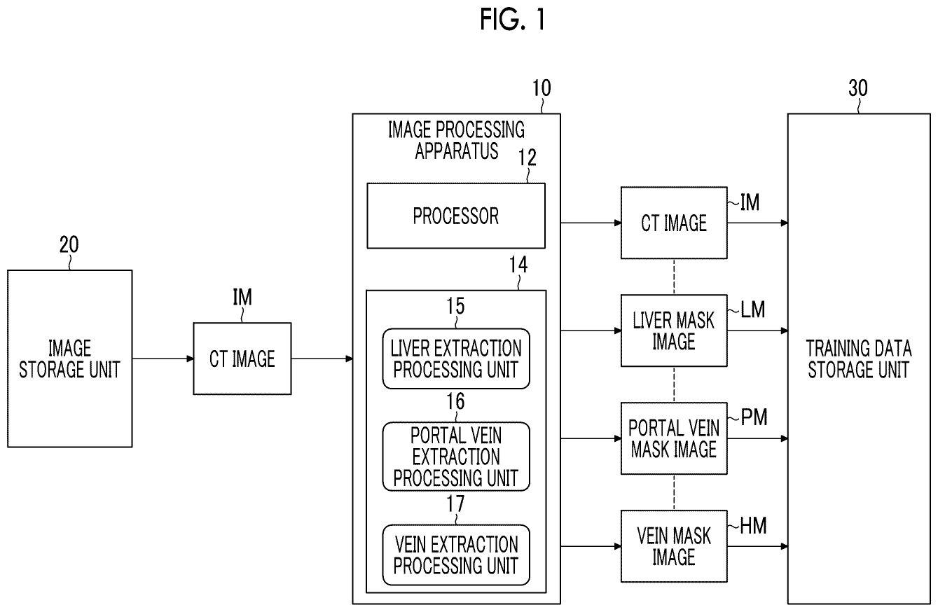

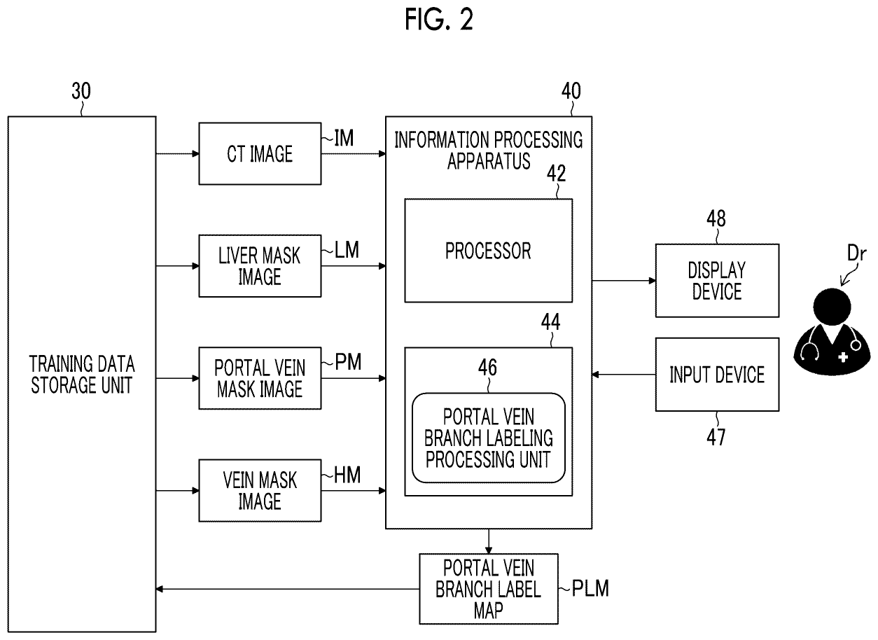

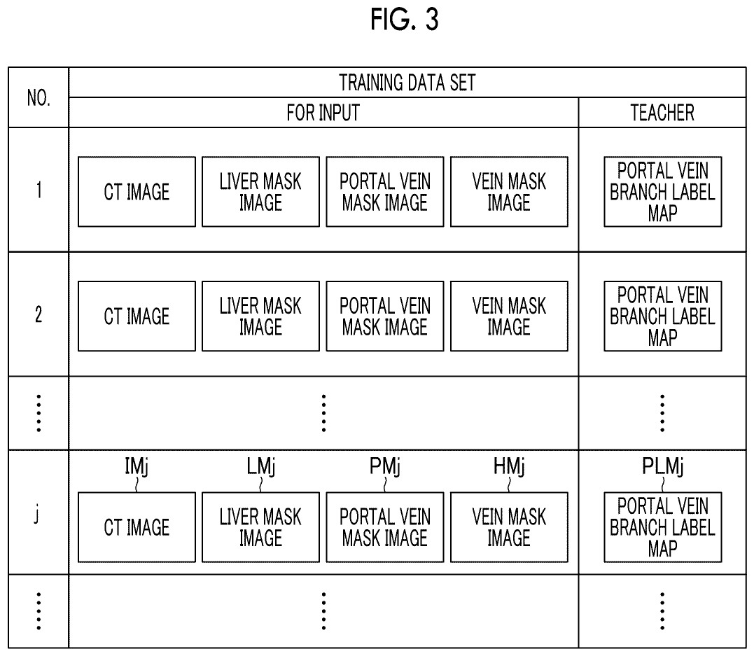

FIGS. 1 and 2 are block diagrams illustrating examples of a method of generating the training data. FIG. 1 illustrates an example of an image processing apparatus 10 that performs processing of generating a liver mask image LM, a portal vein mask image PM, and a vein mask image HM from a CT image IM. FIG. 2 illustrates an example of an information processing apparatus 40 that generates a portal vein branch label map PLM from the CT image IM, the liver mask image LM, the portal vein mask image PM, and the vein mask image HM. In the present embodiment, the training data includes the portal vein branch label map PLM in addition to the CT image IM, the liver mask image LM, the portal vein mask image PM, and the vein mask image HM (refer to FIG. 3).

The CT image IM is a three-dimensional image reconstructed from three-dimensional data obtained by consecutively capturing two-dimensional slice tomographic images. Similarly, each of the liver mask image LM, the portal vein mask image PM, and the vein mask image HM is the three-dimensional image. Note that the term “image” includes meaning of image data.

The image processing apparatus 10 is realized using software and hardware of a computer. The software is synonymous with a program. The image processing apparatus 10 includes a processor 12 and a tangible non-transitory computer-readable medium 14. The form of the image processing apparatus 10 is not particularly limited, and may be a server, a workstation, or a personal computer.

The processor 12 includes a central processing unit (CPU). The processor 12 may include a graphics processing unit (GPU). The computer-readable medium 14 includes a memory as a main storage device and a storage as an auxiliary storage device. For example, the computer-readable medium 14 may be a semiconductor memory, a hard disk drive (HDD) device, or a solid state drive (SSD) device or a combination of a plurality thereof.

The computer-readable medium 14 stores a plurality of programs including an image processing program, data, and the like. The processor 12 functions as a liver extraction processing unit 15, a portal vein extraction processing unit 16, and a vein extraction processing unit 17 by executing a command of a program stored in the computer-readable medium 14.

The liver extraction processing unit 15 performs processing of extracting a region of the liver from the input CT image IM. The liver mask image LM is generated by the liver extraction processing unit 15. The liver mask image LM is an image in which a liver region is specified, and may be, for example, a binary image in which a voxel value of the liver region in the CT image IM is “1” and a voxel value of a region (non-liver region) other than the liver region is “0”.

The portal vein extraction processing unit 16 performs processing of extracting a region of the portal vein from the input CT image IM. The portal vein mask image PM is generated by the portal vein extraction processing unit 16. The portal vein mask image PM is an image in which a portal vein region is specified, and may be, for example, a binary image in which a voxel value of the portal vein region in the CT image IM is “1” and a voxel value of a region (non-portal vein region) other than the portal vein region is “0”.

The vein extraction processing unit 17 performs processing of extracting a region of the vein from the input CT image IM. The vein mask image HM is generated by the vein extraction processing unit 17. The vein mask image HM is an image in which a vein region is specified, and may be, for example, a binary image in which a voxel value of the vein region in the CT image IM is “1” and a voxel value of a region (non-vein region) other than the vein region is “0”.

Each of the liver extraction processing unit 15, the portal vein extraction processing unit 16, and the vein extraction processing unit 17 may be configured to extract each region of the liver, the portal vein, or the vein using the trained model, which is trained to generate the mask image from the input image by machine learning represented by deep learning, for example. The model that performs such an image recognition task is realized using, for example, a CNN such as V-net.

The image processing apparatus 10 acquires the CT image IM from an image storage unit 20, and generates the liver mask image LM, the portal vein mask image PM, and the vein mask image HM corresponding to the CT image IM. The image generated by the image processing apparatus 10 is stored in a training data storage unit 30 in association with the original CT image IM.

Note that FIG. 1 illustrates an example in which the image processing apparatus 10 generates three types of mask images of the liver mask image LM, the portal vein mask image PM, and the vein mask image HM, but the mask images generated by the image processing apparatus 10 are not limited thereto. For example, the image processing apparatus 10 may generate other mask images such as an inferior vena cava mask image in which an inferior vena cava region is specified. In addition, the image processing apparatus 10 may be configured to generate only some of the plurality of types of mask images illustrated in FIG. 1, and may be configured to generate only the portal vein mask image PM, for example.

The image storage unit 20 includes a large capacity storage in which a large number of images including the CT image IM are stored. The image storage unit 20 may be a Digital Imaging and Communications in Medicine (DICOM) server on a network within a medical institution, for example. The DICOM server may be a server operating according to DICOM specifications. The DICOM server is a computer that stores and manages various kinds of data including images captured using CT devices and other modalities, and includes a large-capacity external storage device and a program for database management. The image processing apparatus 10 can acquire a plurality of CT images IM from the image storage unit 20 via a communication line (not illustrated).

The training data storage unit 30 includes a large capacity storage in which data to be used for training is stored. The training data storage unit 30 may be included in the image processing apparatus 10. In addition, a part of a storage area of the image storage unit 20 may be used as the training data storage unit 30.

Next, an example will be described in which the portal vein branches are labeled for the portal vein mask image PM generated by the image processing apparatus 10.

FIG. 2 illustrates an example of the information processing apparatus 40. The information processing apparatus 40 performs the labeling of the portal vein branches on the portal vein region of the portal vein mask image PM. The labeling work is performed by a doctor Dr using the information processing apparatus 40, for example. The information processing apparatus 40 may be a computer including a processor 42 and a tangible non-transitory computer-readable medium 44. The hardware configuration of the processor 42 and the computer-readable medium 44 may be similar to the corresponding elements of the processor 12 and the computer-readable medium 14 described in FIG. 1.

The form of the information processing apparatus 40 may be a server, personal computer, a workstation, a tablet terminal, or the like. For example, the information processing apparatus 40 may be a viewer terminal for image interpretation.

An input device 47 and a display device 48 are connected to the information processing apparatus 40. The input device 47 is configured by, for example, a keyboard, a mouse, a multi-touch panel, or other pointing devices, or an audio input device, or an appropriate combination thereof. The display device 48 is configured by, for example, a liquid crystal display, an organic electro-luminescence (OEL) display, or a projector, or an appropriate combination thereof.

The information processing apparatus 40 can acquire data stored in the training data storage unit 30, and display the data on the display device 48. For example, the information processing apparatus 40 displays the portal vein mask image PM on the display device 48, and accepts an input of the portal vein branch label from the input device 47. The information processing apparatus 40 can acquire not only the portal vein mask image PM but also the CT image IM, the liver mask image LM, and the vein mask image HM, and display the image on the display device 48.

The computer-readable medium 44 stores a plurality of programs including a program that performs labeling of the portal vein branches on the portal vein region of the portal vein mask image PM, the data, and the like. The processor 42 functions as a portal vein branch labeling processing unit 46 by executing a command of the program stored in the computer-readable medium 44.

The portal vein branch labeling processing unit 46 generates the portal vein branch label map PLM on the basis of information (label information) regarding the portal vein branch label input to the input device 47 by the doctor Dr. As described above, the portal vein branch label is a label for classifying a predetermined image region into eight regions. The predetermined image region is the portal vein region. Therefore, the label information is information specifying which portal vein branch each of a plurality of portal vein branch regions, which are a plurality of partial regions included in the portal vein region in the liver, corresponds to. The portal vein branch labeling processing unit 46 accepts an input of the label information, and generates the portal vein branch label map PLM as the teaching data on the basis of the input label information.

The portal vein is classified into eight classes of portal vein branches from S1 to S8, corresponding to the respective hepatic segments S1 to S8. That is, the portal veins are classified such that the portal vein belonging to the S1 hepatic segment is an S1 portal vein branch, the portal vein belonging to the S2 hepatic segment is an S2 portal vein branch, and the like. Therefore, in the label information, the portal vein branch labels S1 to S8 are defined for classifying the portal vein region into eight portal vein branch regions corresponding to the hepatic segments. A table in which the correspondence relationship between portal vein branch labels and hepatic segment labels is defined may be created. It is also possible to interpret the portal vein branch labels by directly replacing the portal vein branch labels with the hepatic segment labels.

The doctor Dr who is the user performs work to assign the portal vein branch label to each portal vein branch region of the portal vein region in the image by using the input device 47 while checking the image such as the portal vein mask image PM displayed on the display device 48.

That is, the doctor Dr designates the correspondence between each portal vein branch region and the portal vein branch label using the input device 47. In response to the input, the portal vein branch labeling processing unit 46 assigns the portal vein branch label to each portal vein branch region included in the portal vein region, and generates a portal vein branch label map PLMj. That is, the portal vein branch labeling processing unit 46 generates the portal vein branch label map PLM in which at least one classification label (portal vein branch label) of the eight classes S1 to S8 is assigned to the portal vein branch region, which is a partial region of the portal vein region, according to the information input via the input device 47. In the portal vein branch label map PLMj, the portal vein region is classified into eight classes based on the portal vein branch label, and the portal vein branch region is colored differently for each portal vein branch label. The portal vein branch label map PLMj may be understood as an image such as a portal vein branch segmentation image.

The portal vein branch label map PLM is stored in the training data storage unit 30 in association with the portal vein mask image PM that is the generation source, on the basis of the information input via the input device 47. In addition, the portal vein branch label map PLM is stored in the training data storage unit 30 in association with the original CT image IM.

Note that, in FIGS. 1 and 2, a case has been described in which the image processing apparatus 10 and the information processing apparatus 40 are separate apparatuses, but the processing function of the image processing apparatus 10 and the processing function of the information processing apparatus 40 can be realized by one computer.

FIG. 3 is a conceptual diagram illustrating an example of a training data set stored in the training data storage unit 30. In the training data storage unit 30, a plurality of data sets in which a CT image IMj, a liver mask image LMj, a portal vein mask image PMj, a vein mask image HMj, and the portal vein branch label map PLMj are associated with each other are stored. The subscript “j” represents an index number for distinguishing between a plurality of data sets.

Here, the CT image IMj, the liver mask image LMj, the portal vein mask image PMj, and the vein mask image HMj are prepared as input data, and the portal vein branch label map PLMj is prepared as teaching (ground truth) data corresponding to the input data. In FIG. 3, a plurality of data sets in which the input data and the teaching data are associated with each other are illustrated as the training data set. Specifically, the training data set is data collection including a plurality of data sets in which input data and the portal vein branch label map PLMj corresponding to the input data are associated with each other.

Note that, in the present embodiment, an example will be given in which a combination of four types of images, the CT image IMj, the liver mask image LMj, the portal vein mask image PMj, and the vein mask image HMj is used as the input data. Although using the combination of four types of images is one of preferred forms, the combination of images used as the input data is not limited to this example. The input data only needs to include at least one of the CT image IMj and the portal vein mask image PMj.

### <<Description of Learning Phase>>

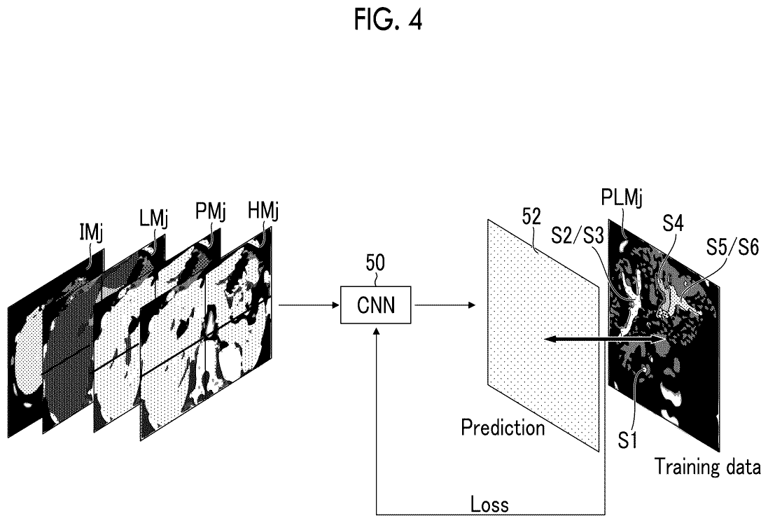

FIG. 4 is a conceptual diagram illustrating an outline of the learning phase. In the learning phase, the machine learning of the learning model 50 is performed on the basis of the input image data, and the trained model 650 is generated. The trained model 650 is applied to a medical image processing apparatus 70 according to the first embodiment. The learning model 50 is configured using the CNN. The learning model 50 may be configured using a neural network based on the V-net architecture, for example.

The learning model 50 is trained to output the portal vein branch label for the predetermined image region on the basis of the input image data (input image). As described above, the portal vein branch label is a label for classifying the predetermined image region into eight regions in association with the eight portal vein branches S1 to S8. Here, as the predetermined image region, the entire region (entire image region) of the input image is classified into eight classes based on the portal vein branch label.

Specifically, the learning model 50 illustrated in FIG. 4 accepts the CT image IMj, the liver mask image LMj, the portal vein mask image PMj, and the vein mask image HMj, as the input image. In addition, the learning model 50 is trained to output the portal vein branch label for each voxel of the entire image region of the input image.

Furthermore, the learning model 50 outputs a score indicating the probability of the portal vein branch label for each voxel of the entire image region of the input image. That is, the learning model 50 outputs the portal vein branch label and the score for each of all the voxels included in the entire image region of the portal vein mask image PMj. The voxel is an example of an “image unit element” in the present disclosure.

That is, the learning model 50 outputs a prediction map 52 indicating the portal vein branch label and the score. The prediction map 52 is a score map of portal vein branch labels in which a score indicating the probability of the portal vein branch label is attached to each voxel of the entire image region. The score map is a probability map that indicates which portal vein branch label from S1 to S8 each voxel is most likely to be, and may be a map in which the portal vein branch label is predicted for the entire region (entire image region) of the image.

In the present embodiment, the entire image region is classified into eight classes from the S1 portal vein branch to the S8 portal vein branch. Therefore, the prediction map 52 output from the learning model 50 is a probability map for each portal vein branch label from the S1 portal vein branch to the S8 portal vein branch. Note that, in FIG. 4, each image is illustrated as a two-dimensional slice cross-sectional image for convenience of illustration, but the image actually handled is a three-dimensional image.

Anatomically, the portal vein branch label is attached to the partial region of the portal vein region. However, the learning model 50 assigns the score indicating the probability of the portal vein branch label to each voxel in the entire image region, including not only the portal vein region in the input image but also regions other than the portal vein region. On the other hand, in a case of calculating the loss between the prediction map 52 output from the learning model 50 and the portal vein branch label map PLMj that is the teaching data, loss calculation is performed by limiting the target to the portal vein region in the input image, regions other than the portal vein region are ignored, and information other than the portal vein region is not reflected in the loss.

In the portal vein branch label map PLMj as the teaching data, the ground truth label is assigned to the portal vein region. Therefore, in the prediction map 52, only the score predicted for the voxel of the portal vein region is reflected in the loss. On the other hand, in the prediction map 52, scores predicted for the voxels other than the portal vein region are ignored without calculating the loss. In this manner, the loss between the prediction map 52 and the portal vein branch label map PLMj is calculated by limiting the target to the portal vein region only, and parameters of the learning model 50 are updated on the basis of the calculated loss. Note that the loss may also be referred to as an error.

By training the learning model 50 using a plurality of training data sets, the parameters of the learning model 50 are optimized, and the trained model is obtained as a result of the learning.

According to the learning method of the present embodiment, the target region for loss calculation is limited to the portal vein region in the image. However, by using a large number of training data sets, images including the portal vein regions of various shapes are learned. As a result, learning that can cover the entire liver region is performed, and the prediction accuracy of the labeling for each voxel is improved.

The input data in which the CT image IMj, the liver mask image LMj, the portal vein mask image PMj, and the vein mask image HMj are combined is an example of “first input data” in the present disclosure. The portal vein mask image PMj is an example of a “first image” in the present disclosure. The entire image region of the portal vein mask image PMj is an example of a “first image region” in the present disclosure. The portal vein branch label map PLMj is an example of “portal vein branch labeling data” in the present disclosure. The data set including the CT image IMj, the liver mask image LMj, the portal vein mask image PMj, the vein mask image HMj, and the portal vein branch label map PLMj is an example of “training data” in the present disclosure.

### Configuration Example of Learning Device

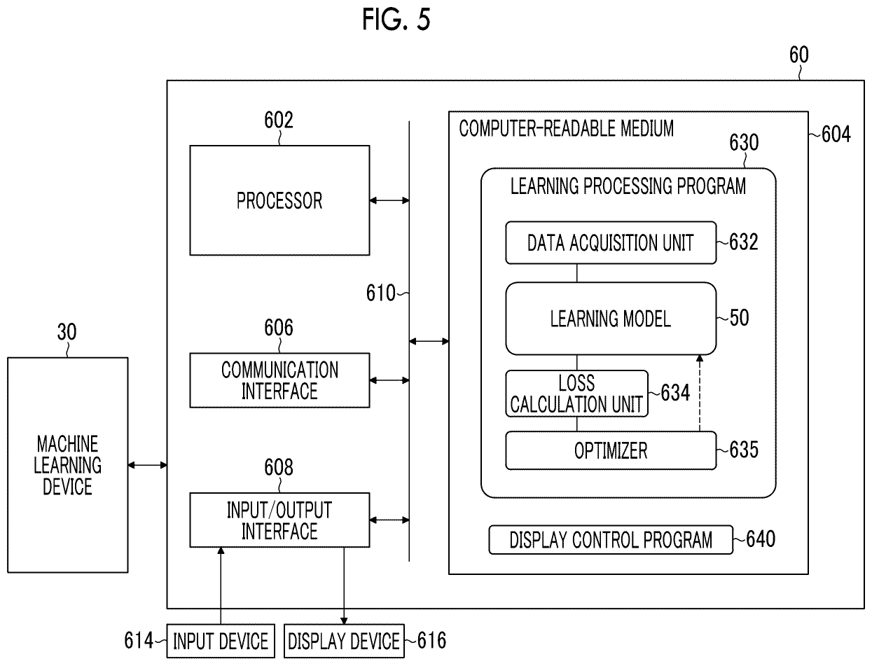

FIG. 5 is a block diagram illustrating a configuration example of a learning device 60. The learning device 60 includes a processor 602, a tangible non-transitory computer-readable medium 604, a communication interface 606, and an input/output interface 608. The hardware configuration of the processor 602 and the computer-readable medium 604 may be similar to the corresponding elements of the processor 12 and the computer-readable medium 14 described in FIG. 1. The form of the learning device 60 may be a server, a personal computer, or a workstation.

The processor 602 is connected to the computer-readable medium 604, the communication interface 606, and the input/output interface 608 via a bus 610. An input device 614 and a display device 616 are connected to the bus 610 via the input/output interface 608.

The hardware configuration of the input device 614 and the display device 616 may be similar to the corresponding elements of the input device 47 and the display device 48 described in FIG. 2. The learning device 60 is connected to a communication line (not illustrated) via the communication interface 606, and is communicably connected to an external device such as the training data storage unit 30.

The computer-readable medium 604 stores a plurality of programs including a learning processing program 630 and a display control program 640, data, and the like. The processor 602 functions as each processing unit of a data acquisition unit 632, the learning model 50, a loss calculation unit 634, and an optimizer 635 by executing commands of the learning processing program 630.

The data acquisition unit 632 acquires training data from the training data storage unit 30. The loss calculation unit 634 calculates the loss between the prediction map 52 and the portal vein branch label map PLM. The portal vein branch label map PLM is teaching data corresponding to the input data used to generate the prediction map 52. The loss calculation unit 634 calculates the loss by limiting the target to the portal vein region where the ground truth label is present in the portal vein branch label map PLM, ignores values of the scores for the voxels of the regions other than the portal vein region, and does not use as the values as the target for the loss calculation. Note that the loss calculation by the loss calculation unit 634 is performed using a loss function, for example.

The optimizer 635 decides an update amount of the parameters of the learning model 50 on the basis of the loss calculated by the loss calculation unit 634, and performs the update processing of the parameters of the learning model 50. The optimizer 635 updates the parameters on the basis of an algorithm such as a gradient descent method. Note that the parameters of the learning model 50 include a filter coefficient (weight of coupling between nodes) of a filter used for processing each layer of the CNN, a bias of the nodes, and the like.

The learning device 60 acquires data from the training data storage unit 30, and executes machine learning of the learning model 50. The learning device 60 can acquire (read) data in units of mini-batch that are a collection of a plurality of training data sets, and update the parameters. In this manner, the learning device 60 generates the trained model 650.

### Example of Learning Method

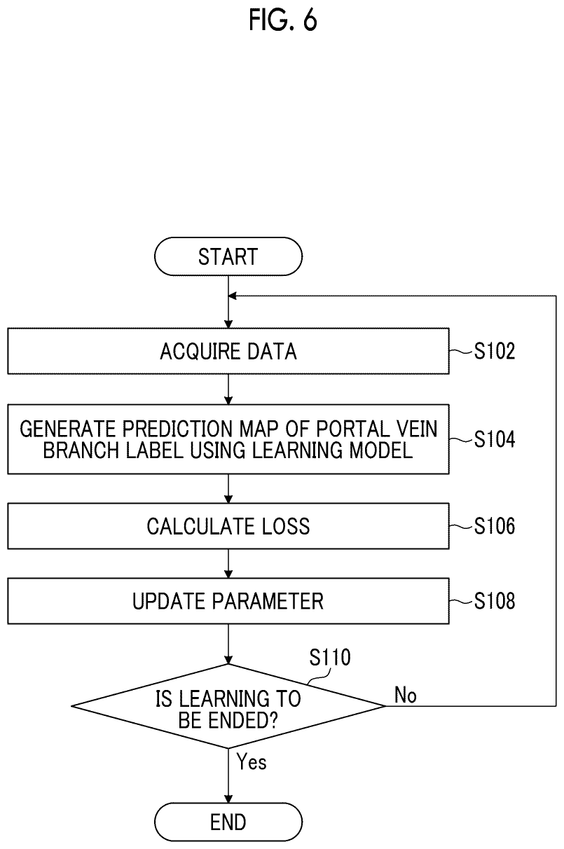

FIG. 6 is a flowchart illustrating a flow of learning processing of the learning device 60. In Step S102, the processor 602 acquires data from the training data storage unit 30. Specifically, the processor 602 accepts an input of training data, and acquires a training data set from the training data storage unit 30.

In Step S104, the processor 602 generates the prediction map 52 of the portal vein branch label using the learning model 50. Specifically, the processor 602 inputs the image (refer to FIG. 3) included in the input data to the learning model 50, and generates the prediction map 52 of the portal vein branch label corresponding to the input data using the learning model 50.

Next, in Step S106, the processor 602 calculates a loss between the prediction map 52 and the portal vein branch label map PLM by limiting the target to the voxel of the portal vein region.

Thereafter, in Step S108, the processor 602 performs the update processing of the parameters of the learning model 50 on the basis of the calculated loss. The operations of Steps S102 to S108 may be performed in units of mini-batch.

In Step S110, the processor 602 determines whether or not to end the learning. A learning end condition may be determined on the basis of the value of the loss, or may be determined on the basis of the number of updates of the parameters. As a method based on the value of the loss, for example, the learning end condition may include that the loss converges within a prescribed range. In addition, as a method based on the number of updates, for example, the learning end condition may include that the number of updates reaches a prescribed number of times.

In a case where No determination is made as a determination result in Step S110, the processor 602 returns to Step S102 and continues the learning processing. On the other hand, in a case where Yes determination is made as the determination result in Step S110, the processor 602 ends the flowchart of FIG. 6.

The trained model is generated by performing the learning method illustrated in the flowchart of FIG. 6. The learning method performed using the learning device 60 is understood as the generation method of the trained model.

### <<Description of Inference Phase>>

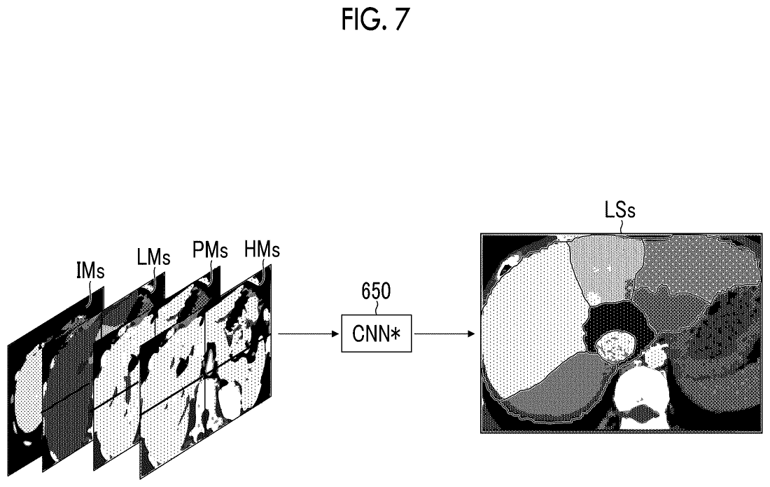

FIG. 7 is a conceptual diagram illustrating an outline of processing in an inference phase using the trained model 650 of the first embodiment. As described above, the trained model 650 is a model obtained such that parameters of the learning model 50 are updated as the result of learning. The inference phase is a phase in which a hepatic segment in image data, which is newly input, is inferred. Specifically, in the inference phase, a hepatic segment division image LSs for a newly input CT image IMs is generated. The hepatic segment division image LSs is generated on the basis of the probability map of the portal vein branch label.

Here, the probability map is a map similar to the prediction map 52 output by the learning model 50. That is, the probability map is also a score map of the portal vein branch label, and is a map in which a score indicating the probability of the portal vein branch label for each voxel included in the entire image region is attached. The probability map is output from the trained model 650. Therefore, the accuracy of the probability map is improved as compared with the prediction map 52.

The hepatic segment division image LSs is a segmentation image in which the liver region of the newly input data is divided into eight hepatic segments. The hepatic segment division image LSs is generated on the basis of the probability map.

The trained model 650 generated by the learning method of the first embodiment receives an input of unknown input data of the same type as the input data used for learning, and generates a score of likelihood of being a portal vein branch label for each voxel in the image. Here, the likelihood of being a portal vein branch label is synonymous with the probability of the portal vein branch label. In addition, the input data of the same type as the input data used for learning is image data in which the region including the liver is imaged, and is data of the CT image and a plurality of types of mask images (refer to input data of FIG. 3). Furthermore, the unknown input data means new image data that has not been used for learning.

Specifically, FIG. 7 illustrates an example of input data of the same type as the input data (refer to FIG. 4) used for learning. A combination of four types of images of a CT image IMs, a liver mask image LMs, a portal vein mask image PMs, and a vein mask image HMs is input to the trained model 650. The subscript “s” is attached to new image data that has not been used for learning and image data obtained as a result of inputting the new image data to the trained model 650.

The liver mask image LMs, the portal vein mask image PMs, and the vein mask image HMs can be generated by performing each of liver extraction processing, portal vein extraction processing, and vein extraction processing on the CT image IMs. These kinds of extraction processing can be performed by processing units similar to the liver extraction processing unit 15, the portal vein extraction processing unit 16, and the vein extraction processing unit 17 described in FIG. 1. The portal vein branch label to which the highest score is attached among the portal vein branch labels assigned to the voxels is adopted on the basis of the probability map of the portal vein branch label. That is, a plurality of portal vein branch labels can be output for each voxel. In addition, the score is output for each of a plurality of portal vein branch labels. In a case where a plurality of portal vein branch labels are output, the portal vein branch label with the highest score among the plurality of portal vein branch labels is adopted as the portal vein branch label of the corresponding voxel. In a case where there is one portal vein branch label attached to the voxel, the portal vein branch label is adopted even though the score is low. Thus, all the voxels included in the entire image region of the input image can be classified into eight classes, and the map in which the portal vein branch label is attached to each voxel in the entire image region can be generated.

The trained model 650 performs label conversion of converting a portal vein branch label into a hepatic segment label corresponding to the portal vein branch label according to the correspondence relationship between the portal vein branch label and the hepatic segment label. In this manner, the liver region can be divided into the hepatic segments on the basis of the portal vein branch label map. Note that the label conversion includes the concept of replacing the portal vein branch label with the hepatic segment label or treating (interpreting) the portal vein branch labels as the hepatic segment label.

Thereafter, only the liver region is extracted from the entire image region. Thus, the hepatic segment division image LSs in which the liver region is divided into the hepatic segments is obtained. The hepatic segment division image LSs is a segmentation image in which the liver region is divided into regions depending on the hepatic segment labels, or a segmentation image in which the liver region is divided into regions depending on the portal vein branch labels interpreted as the hepatic segment labels.

The input data in which the CT image IMs, the liver mask image LMs, the portal vein mask image PMs, and the vein mask image HMs are combined is an example of “second input data” in the present disclosure. The portal vein mask image PMs is an example of a “second image” in the present disclosure. The entire image region of the portal vein mask image PMs is an example of a “second image region” in the present disclosure.

In the first embodiment, a configuration has been exemplified in which, as the input data, four types of images are input to the learning model 50, and the score of likelihood of being a portal vein branch label is output for each voxel of the entire image region of the input image (refer to FIGS. 3 and 4). On the other hand, for example, in a case where, as the input data, only the liver mask image LMj is used in the learning stage, the configuration may be designed such that only the liver region in the image is the learning target. In this case, the learning model 50 may be configured to calculate the score indicating the likelihood of being a portal vein branch label only for the voxels of the liver region, and not to calculate the score indicating the likelihood of being a portal vein branch label for the voxels other than the liver region. The prediction map 52 output from the learning model 50 only needs to be a map including the score of the likelihood of being a portal vein branch label for each voxel of at least the liver region in the image, and is not required to calculate the score of each voxel for all the voxels in the entire image region.

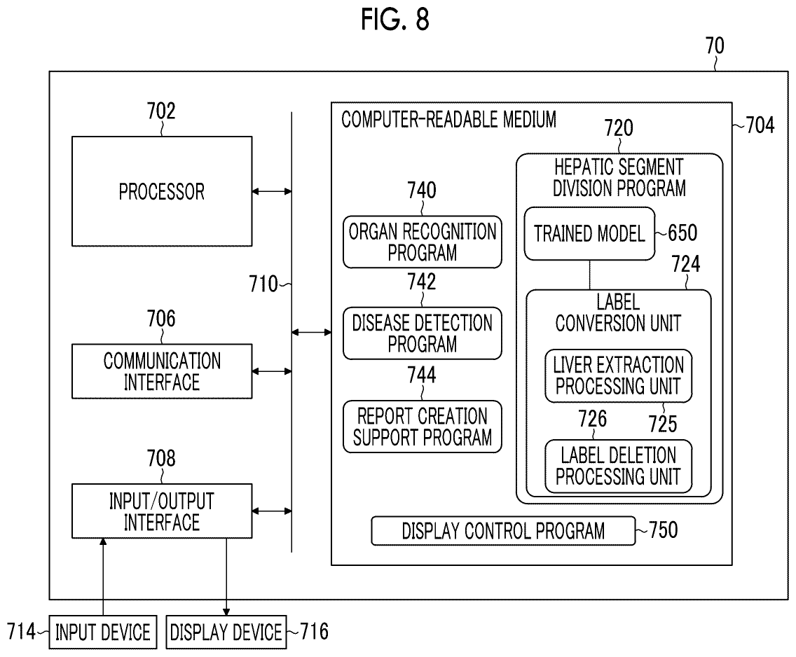

FIG. 8 is a block diagram illustrating a configuration of the medical image processing apparatus 70 according to the first embodiment. The medical image processing apparatus 70 includes a processor 702, a tangible non-transitory computer-readable medium 704, a communication interface 706, an input/output interface 708, and a bus 710. In addition, an input device 714 and a display device 716 are connected to the bus 710 via the input/output interface 708. Each of these elements may be similar to corresponding elements of the processor 602, the computer-readable medium 604, the communication interface 606, the input/output interface 608, the bus 610, the input device 614, and the display device 616 described in FIG. 5.

A form of the medical image processing apparatus 70 is not particularly limited, and may be a server, a personal computer, a workstation, a tablet terminal, or the like. The medical image processing apparatus 70 is connected to a communication line (not illustrated) via the communication interface 706, and is communicably connected to an external device such as the DICOM server.

The computer-readable medium 704 stores a plurality of programs including a hepatic segment division program 720 and a display control program 750, data, and the like. The processor 702 functions as each processing unit of the trained model 650 and a label conversion unit 724 by executing commands of the hepatic segment division program 720. The label conversion unit 724 performs processing of converting a portal vein branch label into a hepatic segment label. That is, the label conversion unit 724 performs labeling of the hepatic segment label on the basis of the portal vein branch label. The label conversion unit 724 may include a liver extraction processing unit 725 that extracts a liver region in the image, and a label deletion processing unit 726 that deletes label information attached to the voxels other than the liver region. The processing algorithm of the liver extraction processing unit 725 may be similar to the liver extraction processing unit 15 described in FIG. 1. Note that, in the present embodiment, the label is invalidated by deleting the label information for the regions other than the liver region, but the present disclosure is not limited thereto, and a form of processing such as masking or ignoring the label information for the regions other than the liver region is also possible.

The computer-readable medium 704 may further include at least one program of an organ recognition program 740, a disease detection program 742, or a report creation support program 744.

The organ recognition program 740 includes a processing module that performs organ segmentation. The organ recognition program may include a lung segment labeling program, a blood vessel region extraction program, a bone labeling program, and the like.

The disease detection program 742 includes a detection processing module corresponding to a specific disease. As the disease detection program 742, for example, at least one of a lung nodule detection program, a lung nodule characteristic analysis program, a pneumonia computer aided diagnosis or computer aided detection (CAD) program, a mammary gland CAD program, a liver CAD program, a brain CAD program, and a large intestine CAD program may be included.

The report creation support program 744 includes a trained document generation model that generates candidates for a finding statement corresponding to a target medical image.

Various processing programs such as the organ recognition program 740, the disease detection program 742, and the report creation support program 744 may be AI processing modules including a trained model that is trained to obtain an output of a target task by applying machine learning such as deep learning.

An AI model for CAD can be configured using, for example, various CNNs having convolutional layers. Input data for the AI model may include, for example, a medical image such as a two-dimensional image, a three-dimensional image, or a video, and an output from the AI model may be, for example, information indicating a position of a disease region (lesion part) in the image, information indicating a class classification such as a disease name, or a combination thereof.

An AI model that handles time series data, document data, and the like can be configured, for example, using various recurrent neural networks (RNNs). In the time series data, for example, waveform data of an electrocardiogram is included. In the document data, for example, a finding statement created by a doctor is included.

The computer-readable medium 704 may further include a program that causes the processor 702 to function as the liver extraction processing unit 15, the portal vein extraction processing unit 16, and the vein extraction processing unit 17 described in FIG. 1. Note that the processing functions of the medical image processing apparatus 70 may be realized by a plurality of computers. In addition, a part or all of the processing functions of the medical image processing apparatus 70 may be incorporated into the image processing apparatus 10 described in FIG. 1.

### Example of Hepatic Segment Division Method

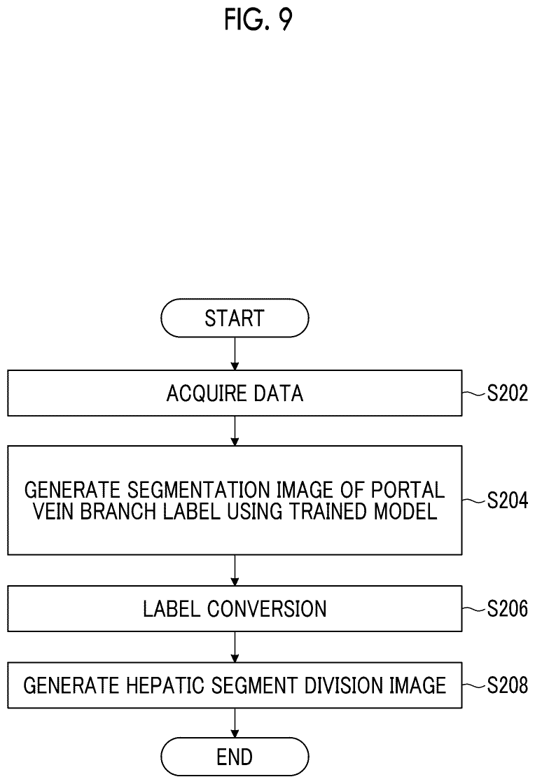

FIG. 9 is a flowchart illustrating an example of a hepatic segment division method using the medical image processing apparatus 70 according to the first embodiment. In Step S202, the processor 702 accepts an input of data including an image of a processing target. In a case where data is input, the processor 702 generates a segmentation image of a portal vein branch label in Step S204. In other words, in Step S204, the probability map of the portal vein branch label is output by the trained model 650. As described above, the probability map is a map in which a portal vein branch label and a score are attached to a predetermined image region.

Specifically, the processor 702 attaches the portal vein branch label and the score to each voxel of the entire image region of the input image or the image region including at least the liver region using the trained model 650. In a case where the portal vein branch label and the score are attached only to each voxel of the liver region, the liver region is the predetermined image region described above. Both the portal vein region and the region other than the portal vein region are included in the entire image region. Furthermore, it is decided which portal vein branch label the voxel corresponding to the predetermined image region belongs to on the basis of the portal vein branch label and the score, and the portal vein branch label is assigned to each voxel. Thus, the segmentation image in which the predetermined image region is classified by the portal vein branch label.

In Step S206, the processor 702 performs processing of label conversion, and divides the liver region into hepatic segments on the basis of the portal vein branch label assigned to each voxel.

In Step S208, the processor 702 generates the hepatic segment division image LSs. Specifically, the processor 702 generates the hepatic segment division image LSs by performing visualization processing such as color-coding each divided hepatic segment to clearly indicate the region. The generated hepatic segment division image LSs can be displayed on the display device 716, a viewer terminal (not illustrated), and the like.

After Step S208, the processor 702 ends the flowchart of FIG. 9.

### Advantages of First Embodiment

With the medical image processing apparatus 70 according to the first embodiment, regardless of the visibility of blood vessels in the CT image IMs, it is possible to accurately divide the liver region in the CT image IMs into hepatic segments.

### Modification Example

There may be various forms of input data used during learning. For example, the input data used during learning may be a combination of three types of masks of the liver mask image LM, the portal vein mask image PM, and the vein mask image HM. In addition, the input data used during learning may be a combination of two types of masks including at least the liver mask image LM.

As the input data used during learning, only the CT image IM (only one type) may be used. In this case, the CT image is the “first image” in the present disclosure. Note that, in this case as well, the “first image region” is the entire image region of the portal vein mask image. Specifically, the entire image region of the portal vein mask image, which is generated from the CT image and has the same image region as the image region of the CT image, is the “first image region”.

In addition, as the input data used during learning, only the portal vein mask image PM may be used. Hereinafter, in a second embodiment, an example will be described in which only the portal vein mask image PM is used as the input data.

### Second Embodiment

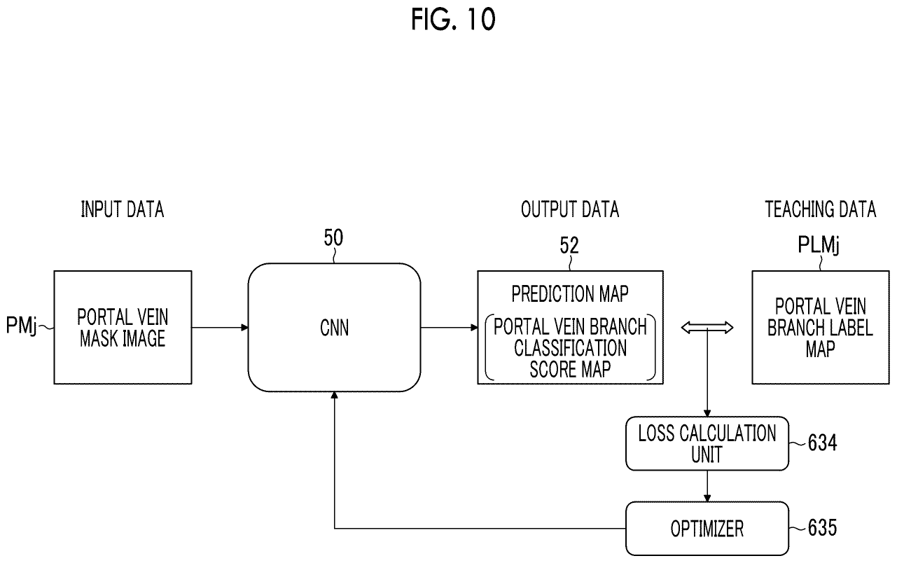

FIG. 10 is a conceptual diagram illustrating an outline of a learning phase in the second embodiment. In FIG. 10, elements that are the same as or similar to those illustrated in FIGS. 4 and 5 are denoted by the same reference numerals, and redundant descriptions thereof will be omitted.

As illustrated in FIG. 10, in the second embodiment, there is only one type of image to be used as the input data to the learning model 50, which is the portal vein mask image PMj. The other processing is the same as that of the first embodiment.

### Preparation of Training Data

For example, the training data used in the second embodiment is prepared as follows.

First, using the image processing apparatus 10 described in FIG. 1 or the like, the extraction processing of the portal vein region is performed on the CT image IMj, and the portal vein mask image PMj is generated as the extraction result.

Then, using the information processing apparatus 40 described in FIG. 2 or the like, the doctor Dr performs labeling of each portal vein branch region by attaching the portal vein branch label to the portal vein region of the same CT image IMj. As a result, the portal vein branch label map PLMj is generated as teaching data.

Thereafter, the portal vein mask image PMj and the portal vein branch label map PLMj are associated, and a data set of the portal vein mask image PMj and the portal vein branch label map PLMj is obtained. By performing similar processing on a large number of CT images, a sufficient number of data sets of the training data sets are prepared.

### <<Description of Learning Processing>>

After the training data set is prepared, learning processing is performed using the learning device 60 described in FIG. 5 or the like. Specifically, learning is performed such that, in a case where the portal vein mask image PMj is input to the learning model 50, a labeling result of the portal vein branch label is output for each voxel of the entire image region including both the portal vein region and the region other than the portal vein region. As in the first embodiment, in the learning processing, the loss is calculated only for the portal vein region with the region other than the portal vein region being excluded from the loss calculation target, and the parameters of the learning model 50 are updated on the basis of the calculated loss.

The portal vein mask image PMj in the second embodiment is an example of the “first input data” and the “first image” in the present disclosure.

### <<Description of Inference Phase>>

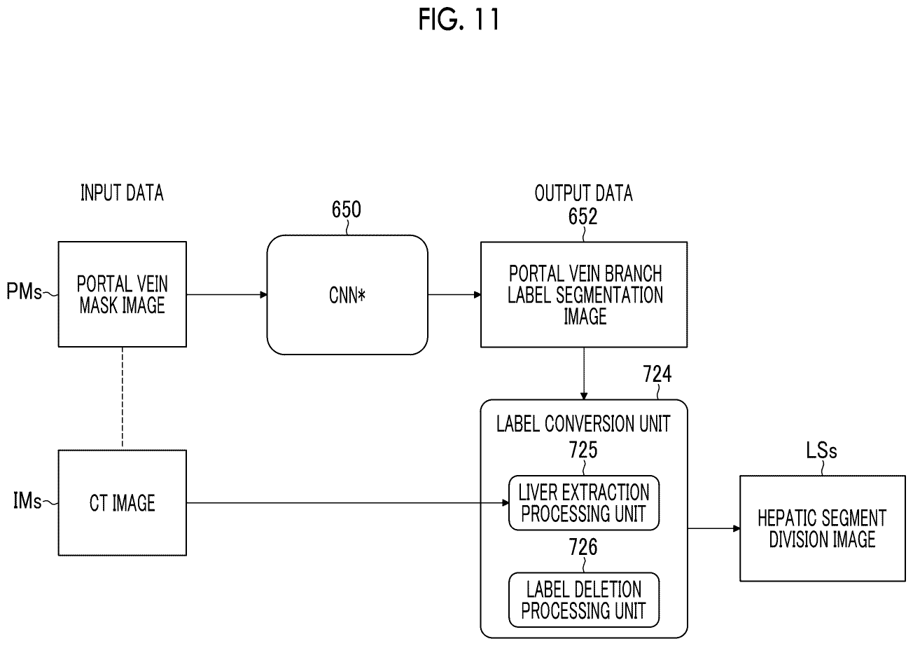

FIG. 11 is a block diagram illustrating an outline of an inference phase using the trained model 650 generated by a learning method of the second embodiment. In FIG. 11, elements that are the same as or similar to those illustrated in FIGS. 7 and 8 are denoted by the same reference numerals, and redundant descriptions thereof will be omitted.

The configuration of a medical image processing apparatus according to the second embodiment may be similar to the configuration of the medical image processing apparatus 70 described in FIG. 7.

In the second embodiment, the trained model 650 is used as follows, for example.

[Procedure 1] The processor 702 first performs extraction of the portal vein region on the CT image IMs including the liver obtained by imaging a patient using the CT device, and generates the portal vein mask image PMs as the extraction result.

[Procedure 2] The processor 702 inputs the portal vein mask image PMs to the trained model 650.

[Procedure 3] The processor 702 uses the trained model 650 to attach the portal vein branch label to the entire region of the input portal vein mask image PMs for each voxel. In addition, in the present embodiment, as in the first embodiment, the trained model 650 decides the portal vein branch label with the highest score among the eight classes of the portal vein branch labels as the portal vein branch label of the corresponding voxel. Specifically, the trained model 650 assigns a plurality of portal vein branch labels for each voxel included in the entire image region of the portal vein mask image PMs. In addition, the score is output for each of the plurality of portal vein branch labels. No matter how low the score indicating the probability predicted for each voxel is, the trained model 650 decides the portal vein branch label with the highest score indicating the probability among the eight classes of the portal vein branch labels as the portal vein branch label of the corresponding voxel. Thus, the portal vein branch labels are assigned to all the voxels in the image. The map indicating the labeling result of the portal vein branch label generated by the trained model 650 is referred to as a portal vein branch label segmentation image 652.

[Procedure 4] The processor 702 attaches the hepatic segment label corresponding to the portal vein branch label on the original CT image IMs on the basis of the portal vein branch label segmentation image 652 generated by the trained model 650.

[Procedure 5] The processor 702 extracts the liver region from the original CT image IMs. The label attached to the region other than the portal vein region is unnecessary. Therefore, the processor 702 deletes the label attached to the region other than the portal vein region. As a result, among the portal vein branch labels attached to the entire image region of the portal vein mask image PMs, only the label attached to the portal vein region remains.

[Procedure 5] Furthermore, the processor 702 performs post-processing such as fine correction of the inference result as necessary. For example, the post-processing here includes processing of filling an isolated small region with a label of a surrounding large region, so-called hole-filling processing. The definition of the small region may be a region having a volume equal to or less than a predetermined volume, for example. The medical image processing apparatus 70 has a configuration of a processing unit that performs fine correction of the labeling result.

In this manner, the liver region is divided into eight classes of hepatic segments from S1 to S8 on the basis of the output data of the trained model 650, and the hepatic segment division image LSs is generated. The hepatic segment division image LSs may be a segmentation image in which the liver region is classified depending on the hepatic segment labels.

According to the second embodiment, the same effects as the first embodiment can be obtained.

### COMPARATIVE EXAMPLE

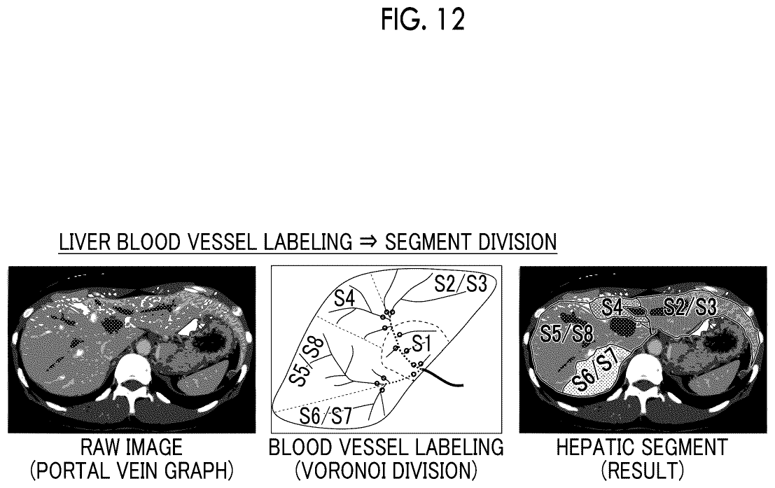

FIG. 12 is an image example illustrating an example of a hepatic segment division method using Voronoi division according to a comparative example. An image illustrated on the left side of FIG. 12 is an example of the CT image from which the portal vein region is extracted. An image illustrated at the center of FIG. 12 is an example of a blood vessel labeling diagram illustrating the portal veins to which the labels are attached by the user designating the branch points of the portal vein branches. An image illustrated on the right side of FIG. 12 is an example of an image illustrating the result of the segment division of the liver region using the Voronoi division based on the blood vessel labeling.

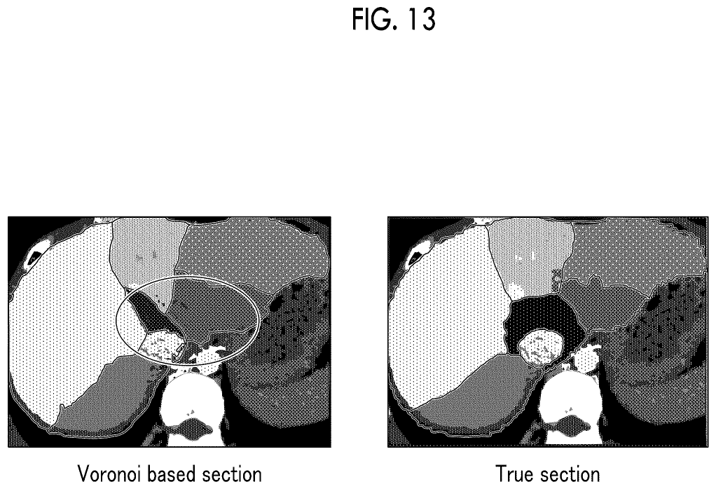

FIG. 13 is a diagram illustrating a comparison between the processing result of the hepatic segment division based on the Voronoi division according to the comparative example and the result of proper hepatic segment division.

An image illustrated on the left side of FIG. 13 is an example of an image illustrating the processing result of the hepatic segment division based on the Voronoi division according to the comparative example, and an image illustrated on the right side of FIG. 13 is an example of an image illustrating the result of the correct (ground truth) hepatic segment division. The division result based on the Voronoi division does not correctly perform the segment division regarding the S1 segment surrounded by a circle. This is because the blood vessels are not completely visible in the CT image, and the S1 portal vein branch cannot be correctly extracted in the stage of the blood vessel labeling.

In this regard, by using the trained model 650 generated using the learning method described in the first and second embodiments of the present disclosure, regardless of the visibility of the blood vessels, it is possible to accurately perform the hepatic segment division.

### <<Regarding Program Operating Computer>>

A program that causes a computer to realize the processing functions of each of the image processing apparatus 10, the information processing apparatus 40, the learning device 60, and the medical image processing apparatus 70 can be recorded on a computer-readable medium as a tangible non-transitory information storage medium such as an optical disk, a magnetic disk, or a semiconductor memory, and the program can be provided via the information storage medium.

In addition, instead of storing and providing the program in a tangible non-transitory computer-readable medium, it is also possible to provide a program signal as a download service by using an electric telecommunication line such as the Internet.

### <<Regarding Hardware Configuration of Each Processing Unit>>

The hardware structure of the processing unit that executes various kinds of processing, such as the liver extraction processing unit 15, the portal vein extraction processing unit 16, and the vein extraction processing unit 17 of the image processing apparatus 10, the portal vein branch labeling processing unit 46 of the information processing apparatus 40, the data acquisition unit 632, the loss calculation unit 634, and the optimizer 635 of the learning device 60, and the label conversion unit 724, the liver extraction processing unit 725, and the label deletion processing unit 726 of the medical image processing apparatus 70, is the following various processors, for example.

The various processors include, for example, a CPU that is a general-purpose processor which executes a program to function as various processing units, a GPU that is a processor specialized for image processing, a programmable logic device (PLD) that is a processor of which the circuit configuration can be changed after manufacture, such as a field-programmable gate array (FPGA), and a dedicated electric circuit that is a processor having a dedicated circuit configuration designed to execute a specific process, such as an application specific integrated circuit (ASIC).

One processing unit may be configured by one processor among these various processors, or may be configured by two or more same or different kinds of processors. For example, one processing unit may be configured by a plurality of FPGAs, a combination of a CPU and a FPGA, or a combination of a CPU and a GPU. Further, a plurality of processing units may be configured by one processor. As an example where a plurality of processing units are configured by one processor, first, there is a form where one processor is configured by a combination of one or more CPUs and software as typified by a computer, such as a client or a server, and this processor functions as a plurality of processing units. Second, there is a form where a processor fulfilling the functions of the entire system including a plurality of processing units by one integrated circuit (IC) chip as typified by a system on chip (SoC) or the like is used. In this manner, various processing units are configured by using one or more of the above-described various processors as hardware structures.

Furthermore, the hardware structures of these various processors are more specifically electrical circuitry where circuit elements, such as semiconductor elements, are combined.

### <<Regarding Types of Medical Images>>

The technology of the present disclosure can use, as the target, various medical images captured by various medical equipment (modalities) without being limited to the CT image. Various medical images include an MR image captured using a magnetic resonance imaging (MRI) device, an ultrasound image that projects human body information, a positron emission tomography (PET) image captured using a PET device, an endoscopic image captured using an endoscope device, and the like. The image as the target of the technology of the present disclosure is not limited to the three-dimensional image, and may be a two-dimensional image. Noted that, in a case of a configuration in which the two-dimensional image is handled, the “voxel” in the contents described in each embodiment described above is replaced with a “pixel” and applied.

### <<Others>>

In the embodiments of the present invention described above, changes, additions, or deletions of the configurations can be appropriately made without departing from the gist of the present invention. The present invention is not limited to the embodiment described above, and many modifications can be made by those skilled in the art within a technical scope of the present invention.

### EXPLANATION OF REFERENCES

10: image processing apparatus
12: processor
14: computer-readable medium
15: liver extraction processing unit
16: portal vein extraction processing unit
17: vein extraction processing unit
20: image storage unit
30: training data storage unit
40: information processing apparatus
42: processor
44: computer-readable medium
46: portal vein branch labeling processing unit
47: input device
48: display device
50: learning model
52: prediction map
60: learning device
70: medical image processing apparatus
602: processor
604: computer-readable medium
606: communication interface
608: input/output interface
610: bus
614: input device
616: display device
630: learning processing program
632: data acquisition unit
634: loss calculation unit
635: optimizer
640: display control program
650: trained model
652: portal vein branch label segmentation image
702: processor
704: computer-readable medium
706: communication interface
708: input/output interface
710: bus
714: input device
716: display device
720: hepatic segment division program
724: label conversion unit
725: liver extraction processing unit
726: label deletion processing unit
740: organ recognition program
742: disease detection program
744: report creation support program
750: display control program
Dr: doctor
IM, IMj, IMs: CT image
HM, HMj, HMs: vein mask image
PM, PMj, PMs: portal vein mask image
LM, LMj, LMs: liver mask image
PLM, PLMj: portal vein branch label map
LSs: hepatic segment division image
S102 to S110: steps of processing of learning method
S202 to S208: steps of processing of hepatic segment division method

What is claimed is:

1. A medical image processing apparatus comprising:
a processor; and
a storage device that stores a program to be executed by the processor,
wherein the program includes
a trained model generated by performing machine learning using training data that includes first input data including a first image regarding a liver, and portal vein branch labeling data in which a portal vein branch label is attached to a portal vein region in the liver in the first image for each portal vein branch corresponding to a hepatic segment,
the trained model is a model obtained by updating parameters of a learning model trained to output a labeling result of the portal vein branch label for each image unit element of a first image region of the first image by accepting an input of the first input data, and
the processor executes a command of the program to
accept second input data which is a same type of input data as the first input data and includes a second image regarding the liver,
assign the portal vein branch label to each image unit element of a second image region of the second image using the trained model, and
divide a liver region included in the second input data into a plurality of the hepatic segments on the basis of the portal vein branch label assigned to each image unit element of the second image region.

2. The medical image processing apparatus according to claim 1,
wherein the first input data includes at least one of a computed tomography (CT) image in which a region including the liver is imaged or a portal vein mask image in which a portal vein region is specified, and
the first image is the CT image or the portal vein mask image.

3. The medical image processing apparatus according to claim 2,
wherein the first input data includes the CT image and the portal vein mask image.

4. The medical image processing apparatus according to claim 2,
wherein the first input data further includes at least one of a liver mask image in which a liver region is specified, a vein mask image in which a vein region is specified, or an inferior vena cava mask image in which an inferior vena cava region is specified.

5. The medical image processing apparatus according to claim 4,
wherein the first input data includes the portal vein mask image, the liver mask image, and the vein mask image.

6. The medical image processing apparatus according to claim 1,
wherein the first image region is an entire region of the first image, and
the second image region is an entire region of the second image.

7. The medical image processing apparatus according to claim 1,
wherein the portal vein branch label is a label for classifying the portal vein branch into eight classes corresponding to eight types of the hepatic segments from S1 to S8.

8. The medical image processing apparatus according to claim 1,
wherein the trained model is configured using a convolutional neural network.

9. The medical image processing apparatus according to claim 1,
wherein processing of the machine learning for generating the trained model includes
calculating a loss only for a portal vein region in which the portal vein branch label is attached, in the portal vein branch labeling data corresponding to the first input data, for a score map indicating a probability of the portal vein branch label output from the learning model, and
updating the parameters of the learning model on the basis of the calculated loss.

10. The medical image processing apparatus according to claim 1,
wherein each of the first image and the second image is a three-dimensional image.

11. The medical image processing apparatus according to claim 1,
wherein the processor performs labeling of a hepatic segment label indicating the hepatic segment on the basis of the portal vein branch label assigned to each image unit element of the second image region.

12. The medical image processing apparatus according to claim 11,
wherein the second input data includes a CT image in which a region including the liver is imaged, and
the processor
extracts a liver region from the CT image included in the second input data, and
invalidates label information labeled for a region other than the extracted liver region, in the second image region.

13. The medical image processing apparatus according to claim 11,
wherein the processor generates a hepatic segment division image in which a region is divided into the hepatic segments, by converting the portal vein branch label assigned to each image unit element of the second image region into the hepatic segment label.

14. A hepatic segment division method of allowing a computer to divide a liver region in an image into hepatic segments, the hepatic segment division method comprising:
generating a learning model generated by performing machine learning using training data that includes first input data including a first image regarding a liver, and portal vein branch labeling data in which a portal vein branch label is attached to a portal vein region in the liver in the first image for each portal vein branch corresponding to the hepatic segment;
generating a trained model by updating parameters of the learning model on the basis of a labeling result of the portal vein branch label that is output by the learning model for each image unit element of a first image region of the first image;
accepting second input data which is a same type of input data as the first input data and includes a second image regarding the liver;
assigning the portal vein branch label to each image unit element of a second image region of the second image using the trained model; and
dividing a liver region included in the second input data into a plurality of the hepatic segments on the basis of the portal vein branch label assigned to each image unit element of the second image region.

15. A non-transitory, computer-readable tangible recording medium, which records thereon a program that causes a computer to operate as a medical image processing apparatus, the program comprising:
a trained model generated by performing machine learning using training data that includes first input data including a first image regarding a liver, and portal vein branch labeling data in which a portal vein branch label is attached to a portal vein region in the liver in the first image for each portal vein branch corresponding to a hepatic segment,
wherein the trained model is a model obtained by updating parameters of a learning model trained to output a labeling result of the portal vein branch label for each image unit element of a first image region of the first image by accepting an input of the first input data, and
the program causes the computer to
accept second input data which is a same type of input data as the first input data and includes a second image regarding the liver,
assign the portal vein branch label to each image unit element of a second image region of the second image using the trained model, and
divide a liver region included in the second input data into a plurality of the hepatic segments on the basis of the portal vein branch label assigned to each image unit element of the second image region.
